(19) United States
(12) Patent Application Publication
(10) Pub. No.: US 2009/0270358 A1
(43) Pub. Date: Oct. 29, 2009

(54) PHARMACEUTICAL FORMULATION OF CLAVULANIC ACID

(75) Inventors: Young B. Lee, Clarksburg, MD (US); Deog J. Kim, Rockville, MD (US); Chang H. Ahn, Potomac, MD (US); Edward C. Scholtz, Blue Bell, PA (US)

Correspondence Address: VENABLE LLP, P.O. BOX 34385, WASHINGTON, DC, 20043-9998, US

(73) Assignee: REXAHN PHARMACEUTICALS, INC., Rockville, MD (US)

(21) Appl. No.: 12/258,062

(22) Filed: Oct. 24, 2008

Related U.S. Application Data
(60) Provisional application No. 60/996,079, filed on Oct. 26, 2007.

(51) Int. Cl. A61K 31/424

(52) U.S. Cl. 514/210.06

(57) ABSTRACT

The present invention generally relates to stable pharmaceutical compositions, and methods of making and administering such compositions. In one aspect, the invention features stabilized pharmaceutical compositions that include pharmaceutically active ingredients such as potassium clavulanate or Clavitesse™, preferably in an immediate-release solid dosage form or an extended-release solid dosage form. Also provided are methods for making and using such immediate-release and stabilized compositions or extended-release and stabilized compositions.

### FIELD OF THE INVENTION

The present invention relates to solid oral dosage forms comprising clavulanic acid, pharmaceutically acceptable clavulanic acid salts, salt compositions and derivatives. In particular, the present invention provides immediate release compositions and extended release compositions of potassium clavulanate that are suitable for daily use and which achieve therapeutic levels of clavulanate. The present invention also relates to the processes for their preparation and to their use as medicaments, for example, for treatment of anxiety, depression, sexual dysfunction and neurological disorders.

### BACKGROUND OF THE INVENTION

The name of clavulanic acid is derived from the Streptomyces clavuligerus microorganisms from which clavulanic acid is derived. Clavulanic acid is biosynthetically generated from the amino acid arginine and the sugar glyceraldehyde 3-phosphate.

Clavulanic acid has negligible intrinsic antimicrobial activity, despite sharing the beta-lactam ring that is characteristic of beta-lactam antibiotics. However, the similarity in chemical structure allows the molecule to act as a competitive inhibitor of beta-lactamases secreted by certain bacteria to confer resistance to beta-lactam antibiotics. When given in combination with some beta-lactam antibiotics like ticarcillin or amoxicillin, clavulanic acid can extend the spectrum and enhance the activity of the antibiotic (AHFS, 1991). This synergistic activity is possible because clavulanic acid acts as an irreversible competitive inhibitor of bacterial beta-lactamases that naturally degrade and inactive beta-lactam antibiotics (Brown et al., J Antibiot (Tokyo). 1976, 29:668-669; Reading and Cole, Antimicrob Agents Chemother. 1977, 11:852-857).

Clavulanic acid is commercially available in the United States only in fixed combination with other drugs. For example, commonly prescribed Timentin®, the combination product of clavulanic acid and ticarcillin, is normally given intravenously in doses ranging from 200-300 mg/kg/day (based on ticarcillin content) which corresponds to a dose of clavulanic acid of approximately 7-10 mg/kg/day (AHFS, 1991). There are no reported adverse reactions or contraindications for clavulanic acid given in this dose range (Koyu et al., Jpn J. Antibiot. 1986, 39:2831-2862; Yamabe et al., Chemioterapia. 1987, 6:337-40). Augmentin® (co-amoxiclav), the combination product of clavulanic acid and amoxicillin has shown the effectiveness against amoxicillin-resistant β-lactamase-producing strains. Standard adult dosages for respiratory tract, urinary, abdominal and dental infections as well as cellulitis and animal bites is co-amoxiclav 250/125 (250 mg of amoxicillin/125 mg of clavulanic acid) taken every 8 hours, which may be doubled in severe infections. In the US, Augmentin XR (co-amoxiclav 1000/62.5) is marketed for use in community acquired pneumonia with two tablets taken twice a day

In addition to its inhibitory effect on beta-lactamases, clavulanic acid has shown effectiveness for neuroprotection, and in treating anxiety and sexual dysfunction. Koppel et al., in U.S. Pat. Nos. 6,489,319; 6,610,681; and 6,627,625 describe that clavulanic acid itself has an anxiolytic activity when administered i.p. at less than 1 microgram/kg. In U.S. Pat. No. 6,426,342 Koppel describes the potent neuroprotectant activity of clavulanic acid when treated rats with clavulanic acid at an i.p. dose of 1 μg/kg. Koppel in U.S. Pat. No. 7,166,626 also discloses a method for treating sexual dysfunction with the administration of clavulanic acid. U.S. Pat. No. 6,489,319 reports that clavulanic acid could alter CNS activity and behavior at doses ranging from 10 ng to 10 μg/kg. Thus the unique neurological activity profiles of clavulanic acid provide strong evidence that the compound interacts with unique sets of neurogenic targets. Rothstein et al also demonstrated that several beta-lactam antibiotics could offer neuroprotection by the activation of the gene for glutamate neurotransmitter transporter (Nature, 2005, 433:73-77). Since first identified with the discovery of penicillin in 1928, beta-lactam antibiotics have been among the most widely used antibiotics, but have not shown substantial toxic CNS actions at normal antibacterial doses. Therefore, beta-lactam antibiotics may be used as a new and safe therapeutic agent for the treatment of CNS related diseases.

The preparation of many of dry formulations containing clavulanic acid and derivatives or salts thereof (collectively referred to as clavulanate) has necessitated the inclusion of a complex formulation of excipients, including binders, glidants, disintegrants and even desiccants, etc. to yield a pharmaceutically acceptable carrier. This is in part due to the fact that clavulanate is a highly hygroscopic material which is highly unstable in aqueous media. Methods of formulation must therefore ensure that the product can retain its potency during storage, and yet can subsequently yield satisfactory dissolution rates. One such process is disclosed in WO 92/19227 and mandates the inclusion of both an intra-cellular and an extra-cellular disintegrant. Another process described in U.S. Pat. No. 4,537,887 specifies the inclusion of an edible desiccant within the composition itself. Other processes warrant the inclusion of a desiccant within a container housing the amoxicillin/clavulanate combination. In this regard, U.S. Pat. Nos. 4,301,149 and 4,441,609 are particularly salient.

Potassium clavulanate is more stable than the free acid and the least hygroscopic of the pharmaceutically acceptable clavulanic acid salts, and it is therefore most frequently used for commercial preparations. However, potassium clavulanate is still extremely hygroscopic and susceptible to hydrolysis so that co-amoxicillin/clavulanate formulations are prone to degradation on storage even under low humidity conditions. The presence of water in crystallization of amoxicillin may contribute to instability of these dosage forms, accelerating the decomposition of clavulanate once any degradation has commenced.

### SUMMARY OF THE INVENTION

Clavulanate is an exceptionally difficult material to formulate because of its moisture and heat sensitive properties. There is a need to develop stable formulations of clavulanate alone, especially at low doses such as 10 μg to 10 mg, for example, from about 0.1 mg to about 5 mg, which is orally active and may be used for anxiety, depression, neuroprotection, sexual dysfunction, etc.

The present invention is a stable oral dosage composition containing clavulanate, including an immediate release composition and an extended release composition, prepared from clavulanic acid or derivatives or salts thereof, for example potassium clavulanate or Clavitesse™, that is suitable for daily use.

The present invention overcomes and alleviates the above mentioned drawbacks and disadvantages through the development of novel oral clavulanate pharmaceutical compositions and methods. Generally speaking, the present invention relates to stabilized solid pharmaceutical compositions and in particular, immediate release or extended release, stabilized pharmaceutical compositions that include clavulanate as the pharmaceutically active ingredient. The novel pharmaceutical compositions can be provided in a solid dosage form, such as a tablet, capsule, pill, troche or powder. The solid pharmaceutical composition can include a clavulanate in the presence of one or more pharmaceutically acceptable excipients, where the clavulanate present in an amount of between about 10 μg and about 10 mg or, for example, from about 0.1 mg to about 5 mg. The composition can provide a therapeutically useful amount of clavulanate upon administration. Examples of clavulanates include clavulanic acid, clavulanic acid derivatives and pharmaceutically acceptable salts of clavulanic acid. The clavulanate can be present in an amount between about 0.01% and about 10% by weight of the composition. In some embodiments, the moisture content of the composition is less than about 4% of the total weight. The formulation is the form of a tablet, capsule, pill, troche or powder. Exemplary solid pharmaceutical compositions according to the invention can have a moisture content of less than 10% after storage at 25° C. and 60% relative humidity or after storage at 30° C. at 65% relative humidity for three months.

In exemplary compositions, the clavulanate is potassium clavulanate. The potassium clavulanate can be provided as, for example, a powder or as a 1:1 mixture with silicon dioxide or microcrystalline cellulose. Exemplary compositions are immediate-release compositions which release more than 80% of clavulanate from the tablet within approximately 5 to approximately 30 minutes after administration. In exemplary embodiments, the composition is prepared by a method where potassium clavulanate powder is lyophilized in the presence of the one or more pharmaceutically acceptable excipients. In an example of an immediate release composition, the composition can contain from about 10% to about 20% by weight of a binder or diluent, about 45% to about 55% by weigh of a filler, about 20% to about 40% by weight of a disintegrant and about 3% to about 6% by weight of a lubricant. In a such an embodiment, an exemplary binder or diluent is Maltrin M150, an exemplary filler is Prosolve SMCC 50, an exemplary disintegrant is Pharmaburst and/or L HPC LH-11 and/or Acdisol and an exemplary lubricant is stearic acid.

In other exemplary embodiments, the composition is prepared by a method where potassium clavulanate in a 1:1 mixture with silicon dioxide or microcrystalline cellulose is lyophilized in the presence of the one or more pharmaceutically acceptable excipients. In another example of an immediate release composition, the composition can contain from about 50-60% of a filler, about 20-30% of a disintegrant, about 0.5-5% of a flow enhancer/moisture protectant and/or about 3-6% of a lubricant. In a such an embodiment, an exemplary filler is Prosolve SMCC 50, an exemplary disintegrant is Pharmaburst and/or Acidisol, an exemplary flow enhancer/moisture protectant is Carbosil and an exemplary lubricant is magnesium stearate.

In another embodiment, the pharmaceutical composition is an extended-release composition which releases the potassium clavulanate over at least about 4 hours. An extended release composition can be prepared where a potassium clavulanate powder or a potassium clavulanate in a 1:1 mixture with microcrystalline cellulose is lyophilized in the presence of the one or more pharmaceutically acceptable excipients. Exemplary excipients can include one or more of a matrix, a filler, a glidant and a lubricant. In an example of an extended release composition, the composition can contain from about 20% to about 40% by weight of a matrix, about 50% to about 75% by weight of a filler, about 0.1% to about 1% by weight of a glidant and about 1% to about 2% by weight of a lubricant. In such an embodiment, exemplary matrices are Klucel LF and/or Methocel K100LV Prem-M CR, Eudragit RS PO powder, or mixtures thereof; exemplary fillers are anhydrous lactose, Avicel PH-112, Avicel PH-113, Isomalt, or mixtures thereof; an exemplary glidant is Carbosil and an exemplary lubricant is at least one of magnesium stearate and talc.

In other embodiments, a solid pharmaceutical dosage form is prepared by providing a clavulanate such as clavulanic acid, clavulanic acid derivatives or a pharmaceutically acceptable salt of clavulanic acid; mixing the clavulanate with at least one excipient; granulating the mixture of clavulanate and the at least one excipient; and lyophilizing the granulated mixture of clavulanate and the at least one excipient. The granulating step can be, for example wet granulation. An exemplary clavulanate is potassium clavulanate, for example in the form of potassium clavulanate powder or potassium clavulanate as a 1:1 mixture with silicon dioxide or microcrystalline cellulose. In an exemplary method, the excipient at least one of a binder, a diluent, a filler, a disintegrant, a matrix, a filler, a glidant, a flow enhancer, a moisture protectant, and a lubricant. The method can include forming the dosage form into a tablet or bead, and optionally coating the tablet or beads with a delay-release polymer.

The invention includes treatments such as administering a solid pharmaceutical composition according to the invention in to provide an amount of clavulanate effective for the treatment of a disorder such as sexual dysfunction and neurological disorders. In some embodiments, an extended release composition is utilized a the disorder is anxiety and depression disorder. In other embodiments, an immediate release composition is utilized and the disorder is sexual dysfunction.

Still other embodiments of the present invention relate to immediate and extended release formulations of clavulanate that are suitable for oral administration.

Yet other embodiments of the present invention relate to a freeze drying method for preparing the pharmaceutical formulation, wherein the freeze drying comprises the drying process to dehydrate the hydrated pharmaceutical composition.

Other embodiments of the invention relate to a processes for the preparation of pharmaceutical compositions containing clavulanate and to their use as medicaments.

### BRIEF DESCRIPTION OF THE DRAWINGS

FIG. 1 shows in vitro dissolution profiles of clavulanate immediate release formulation, Sample B () and C (◯).

FIG. 2 shows in vitro dissolution profiles of clavulanate extended release formulation, Sample F.

FIG. 3 shows in vitro dissolution profiles of clavulanate extended-release formulation, Sample I.

FIG. 4 illustrates the stability of Sample D (5 mg/tablet of 1:1 mixture of potassium clavulanate and microcrystalline cellulose) at 25° C./60% humidity () and 30° C./65% humidity (▴).

FIG. 5 illustrates the stability of Sample E (5 mg/tablet of 1:1 mixture of potassium clavulanate and silicon dioxide) at 25° C./60% humidity () and 30° C./65% humidity (▴).

FIG. 6 illustrates the stability of Sample F (5 mg/tablet of 1:1 mixture of potassium clavulanate and microcrystalline cellulose) at 2-8° C. (◯), 25° C./60% humidity () and 30° C./65% humidity (▴).

FIG. 7 illustrates the stability of Sample G (5 mg/tablet) at 2-8° C. (◯), 25° C./60% humidity () and 30° C./65% humidity (▴).

### DETAILED DESCRIPTION OF THE INVENTION

As used herein, the term clavulanate herein includes clavulanic acid (I), pharmaceutically acceptable clavulanic acid salts, salt compositions and derivatives, such as esters. An example of pharmaceutically acceptable clavulanic acid salts is potassium clavulanate. Potassium clavulanate may be supplied as a pure compound or as Clavitesse™, a 1:1 mixture of potassium clavulanate and microcrystalline cellulose or a 1:1 mixture of potassium clavulanate and silicon dioxide (available from DSM Anti-Infectives B.V., The Netherlands).

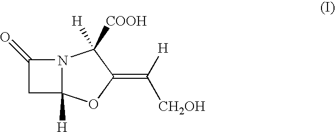

The term “oral administration” as used herein includes any form of delivery of a therapeutic agent or a composition thereof to a subject wherein the agent or composition is placed in the mouth of the subject, whether or not the agent or composition is swallowed. Thus “oral administration” includes buccal and sublingual as well as esophageal administration. Absorption of the agent can occur in any part or parts of the gastrointestinal tract including the mouth, esophagus, stomach, duodenum, ileum and colon.

As used herein, a “subject” to which a therapeutic agent or composition thereof can be administered includes a human patient of either sex and of any age, and also includes any nonhuman animal, particularly a domestic or companion animal, illustratively a cat, dog or horse.

The term “neurological” refers to conditions, disorders, and/or diseases that are associated with the nervous system. Thus, any condition, disorder and/or disease that effect any component or aspect of the nervous system (either central or peripheral) are referred to as a neurological condition, disorder and/or disease. As used herein, the term “neurological” encompasses the terms “neuropsychiatric” or “neuropsychiatry” and “neuropsychological” or “neuropsychology”. Thus, a neurological disease, condition, or disorder includes, but is not limited to cognitive disorders, affective disorders (e.g., depression and/or anxiety disorders), movement disorders, mental disorders, pain disorders, sleep disorders, etc.

The term “excipient” as used herein means any substance, not itself a therapeutic agent, used as a carrier or vehicle for delivery of a therapeutic agent to a subject or added to a pharmaceutical composition to improve its processing, handling, storage, disintegration, dispersion, dissolution, release or organoleptic properties or to permit or facilitate formation of a dose unit of the composition into a discrete article such as a capsule or tablet suitable for oral administration. Excipients can include, by way of illustration and not limitation, diluents, disintegrants, binding agents, adhesives, wetting agents, polymers, lubricants, glidants, substances added to mask or counteract a disagreeable taste or odor, flavors, dyes, fragrances, and substances added to improve appearance of the composition.

The present invention is thus directed to an immediate or extended release formulation of potassium clavulanate or Clavitesse™ which is suitable for oral administration. The formulations of the present invention comprise a quantity of a quick release preparation of clavulanate or a quantity of a slow release (or extended release) preparation of clavulanate. The immediate release formulation of the present invention is characterized by its rapid release of clavulanate, the rapid release characterized by obtaining a maximal release of clavulanate within approximately 5 to approximately 30 minutes after administration. The extended release formulation is characterized by a slower release of clavulanate over, for example, at least about 4 hours. In other exemplary embodiments, the extended release formulation can release clavulanate over at least about 6 or at least about 8 hours. These or other embodiments can continue to release clavulanate after initial administration for at least about 3 hours, at least about 4 hours, at least about 5 hours, at least about 6 hours, at least about 7 hours, or at least about 8 hours. In an exemplary embodiment, the present invention is a tablet or a capsule containing the immediate or extended release formulation, which, based upon the total quantity of drug in the formulation rather than total weight of the formulation, comprises the amount of active compound from about 10 μg to 10 mg or about 0.01% to 10% of total weight of the active compound.

The oral administration of such pharmaceutical agents as tablets or capsules has certain advantages over parenteral administration such as i.v. or i.m. Diseases requiring treatment with painful injectable formulations are considered to be more serious than those conditions which can be treated with oral dosage forms. However, the major advantage with oral formulations is held to be their suitability for self administration whereas parenteral formulations have to be administered in most cases by a physician or paramedical personnel.

The nature of various drug substances, e.g., particle size distribution, bulk density, flowability, wetting behavior, surface area and sticking tendency, varies greatly and can effect the processability of a solid dosage form such as a tablet. Clavulanate is highly hygroscopic and, upon contact with water, changes from a crystalline state to an amorphous state, which shows inferior stability. The combination of these hurdles makes standard tablet manufacturing processes extremely difficult, makes storage of clavulanate formulations problematic, and has resulted in special conditions for storage and preparation of formulations containing clavulanate.

Potassium clavulanate, although the most common and easily handled form, remains an exceptionally difficult material to formulate, being extremely hygroscopic and moisture sensitive. Degradation readily occurs in the presence of water and aqueous media.

Accordingly, a suitable and robust clavulanate formulation overcoming the above problems that takes into account the properties of clavulanate needs to be developed. The problems encountered with clavulanate formulations are particularly challenging in the case of formulations at low dosages such as 10 μg to 10 mg where even a small degree of degradation can lead to a dramatic change in the amount of clavulanate available to a subject.

The present invention relates to the preparation of the stable solid oral dosage forms of Clavulanate and their use in the treatment of sexual dysfunction, depression, or anxiety, or neurological disorders. Solid oral dosage forms according to the invention can comprise additives or excipients that are generally suitable for the preparation of the solid oral dosage form.

Tabletting aids, commonly used in tablet formulation can be used and reference is made to the extensive literature on the subject, see in particular Fiedler's “Lexicon der Hilfstoffe”, 4th Edition, ECV Aulendorf 1996, which is incorporated herein by reference. These include, but are not limited to, fillers, binders, disintegrants, lubricants, glidants, stabilizing agents, fillers or diluents, surfactants, film formers, softeners, pigments and the like.

Fillers include starches, e.g., potato starch, wheat starch, corn starch, hydroxypropyl cellulose, hydroxyethyl cellulose, hydroxypropyl methyl cellulose (HPMC) and, microcrystalline cellulose, e.g., products available under the registered trade marks AVICEL, FILTRAK, HEWETEN, Prosolve SMCC50 or PHARMACEL. Other examples of fillers include lactose, sucrose, glucose, mannitol, sorbitol, and calcium carbonate.

Binders include starches, sugars, cellulose or modified cellulose such as hydroxypropyl cellulose, lactose, or sugar alcohols like xylitol, sorbitol or maltitol. An exemplary binder is maltodextrin (Maltrin M150).

As disintegrants one can mention carboxymethylcellulose calcium (CMC-Ca), carboxymethylcellulose sodium (CMC-Na), crosslinked PVP (e.g. CROSPOVIDONE, POLYPLASDONE or KOLLIDON XL), alginic acid, sodium alginate and guar gum. Crosslinked PVP (CROSPOVIDONE), crosslinked CMC (Ac-Di-Sol), carboxymethylstarch-Na (PIRIMOJEL and EXPLOTAB), Pharmaburst and hydroxypropylcellulose (L HPC LH-11) are exemplary disintegrants.

A matrix can include, for example, Methocel K100 Prem-M or Eudragit RS PO powder.

Examples of glidants include colloidal silica, such as colloidal silicon dioxide, e.g., fumed silica (Cabosil, Aerosil), magnesium (Mg) trisilicate, powdered cellulose, starch, talc and tribasic calcium phosphate or combinations of these with fillers or binders, e.g., silicified microcrystalline cellulose (PROSOLV). Cabosil can also function as a flow enhancer/moisture protecting agent.

Further, fillers or diluents can include confectioner's sugar, compressible sugar, dextrates, dextrin, dextrose, lactose, mannitol, microcrystalline cellulose, for example microcrystalline cellulose having a density of about 0.45 g/cm³, such as AVICEL, powdered cellulose, sorbitol, sucrose and talc.

Lubricants include stearic acid and salts thereof, such as magnesium stearate, aluminum stearate, and calcium stearate, PEG 4000 to PEG8000, talc, hydrogenated castor oil, glycerol esters, Na-stearylfumarate, hydrogenated cotton seed oil and others. A common lubricant are stearic acid and Mg stearate.

Tablets and capsules can additionally be prepared with enteric coatings and other release-controlling coatings for the purpose of light protection, and swallowability. Examples of enteric coatings may include compounds prepared from, for example, methacrylic acid copolymers, cellulose acetate (and its succinate and phthalate version), styrol maleic acid co-polymers, polymethacrylic acid/acrylic acid copolymer, hydroxypropyl methyl cellulose phthalate, polyvinyl acetate phthalate, hydroxyethyl ethyl cellulose phthalate, hydroxypropyl methyl cellulose acetate succinate, cellulose acetate tetrahydrophthalate, acrylic resin, timellitate, and shellac. Exemplary polymers for enteric coatings include methacrylic copolymers such as Eudragit. Other suitable polymers for enteric coatings are known in the art. The coating may be colored with a pharmaceutically accepted dye. The amount of dye and other excipients in the coating liquid may vary and will not impact the performance of the immediate or extended release tablets. The coating liquid generally comprises film forming polymers such as hydroxy-propyl cellulose, hydroxypropylmethyl cellulose, cellulose ester or ether, an acrylic polymer or a mixture of polymers. The coating solution is generally an aqueous solution further comprising propylene glycol, sorbitan mono-oleate, sorbic acid, fillers such as titanium dioxide, a pharmaceutically acceptable dye.

Solid oral dosage forms according to the present invention comprise a therapeutically effective amount of clavulanate as an active agent, and a filler as an additive. Further additives can include, but are not limited to, binders, disintegrants, lubricants, glidants, stabilizing agents, diluents, surfactants, film formers, pigments, softeners and antitacking agents and the like.

Potassium clavulanate is both hygroscopic and readily hydrolyzed by water, so for handling and long term storage of potassium clavulanate it is generally necessary for the immediate environment to be kept extremely dry. This has been accomplished in the past by adding edible silicon dioxide to a composition or by storage of a composition in the presence of a desiccant within a sealed container.

Potassium clavulanate has relatively low moisture content (<1% on a dry weight basis) when exposed to about 35% of relative humidity for 96 hr as shown in Table 7. However, it appears that deliquescence would eventually occur at any humidity above 40% relative humidity. Moisture absorption by dry potassium clavulanate exposed to 50% relative humidity occurs at a rate of approximately 1.44% per hour.

We have found that the use of lyophilization, or freeze drying, during the preparation of pharmaceutical compositions containing clavulanate increases the stability of the clavulanate tablet to about 97% (See Table 8).

According to the present invention, stable pharmaceutical compositions can be prepared that include clavulanate as the pharmaceutically active ingredient (API) at doses ranging from about 10 μg to 10 mg, for example, from about 0.1 mg to about 5 mg. In an exemplary embodiment, the clavulanate is a clavulanate salt, for example potassium clavulanate. It has been reported that clavulanic acid can alter CNS activity and behavior at doses ranging from 10 ng to 10 μg/kg (See U.S. Pat. No. 6,489,319). Methods for treating sexual dysfunction also include the administration of clavulanic acid at doses ranging from 10 ng to 10 μg/kg (See U.S. Pat. No. 7,166,626).

According to the present invention, various dosage forms of clavulanate can be prepared including immediate release and extended release dosage forms that contain from about 10 μg to about 10 mg clavulanate, for example from about 0.1 mg to about 5 mg clavulanate. Such dosage forms can be used for the treatment of sexual dysfunction, anxiety disorder and symptoms hereof. In particular, the immediate release form in the present invention can be used for the treatment of sexual dysfunction and symptoms thereof. The extended release formulation of this invention can be used for the treatment of anxiety, depression and symptoms thereof.

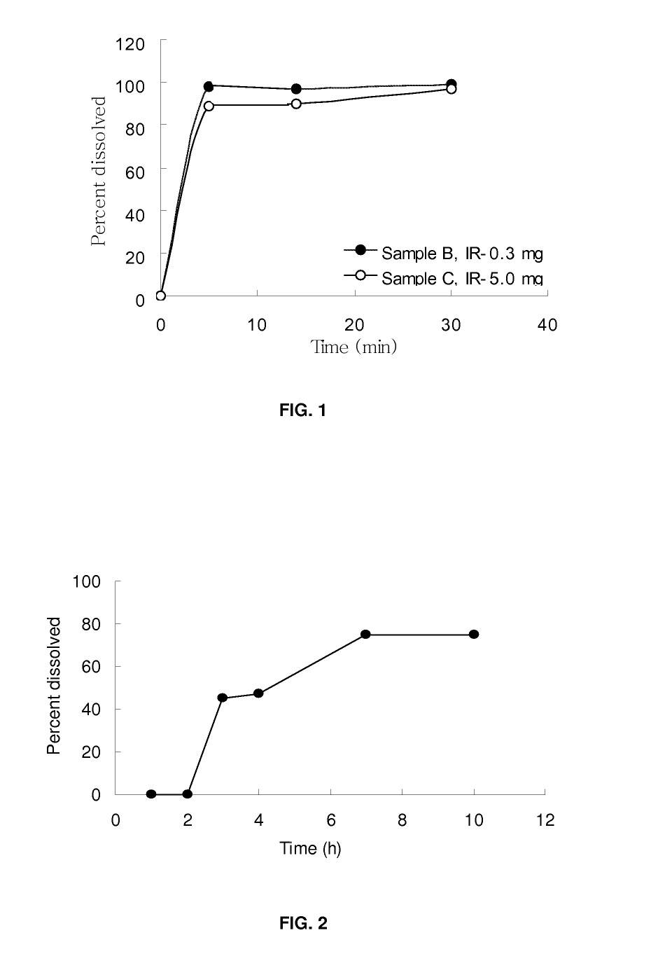

Immediate release forms desirably provide at least about 80% (w/v) dissolution of the clavulanate in less than about 30 minutes as determined by standard assays disclosed herein. The immediate release pharmaceutical compositions according to embodiments of the invention can be rapidly dissolved in an appropriate aqueous solution (e.g., water, saline, juice) or colloidal suspension (e.g., baby formula or milk) for convenient administration to patients unable to handle solid dosage forms. Illustrative of such patients are infants, children, and adults who may experience swallowing difficulties. Accordingly, and in one embodiment, the invention features an immediate release pharmaceutical composition including clavulanate, such as a clavulanate salt. In exemplary embodiments, at least about 80% of the clavulanate is dissolved in aqueous solution by about 15 minutes from the time that the composition is placed in the aqueous solution. In other embodiments, at least about 90% of the clavulanate is released to the aqueous solution by about 30 minutes, or by about 15 minutes, after exposure of the composition to the aqueous solution. As shown in FIG. 1, exemplary immediate release compositions in accordance with the present invention release 90% of the clavulanate within 15 minutes after exposure to an aqueous solution.

Extended release compositions can release the active ingredient, i.e. clavulanate, over a long period, for example over about 8 hours or over about 10 hours. An extended release formulation can begin releasing the active ingredient as soon as the formulation reaches gastrointestinal track and continue to dissolve slowly and release the active ingredient in an approximately constant manner. This profile is desired because it provides steadier levels of the active ingredient in the bloodstream after administration. As shown in FIG. 2, exemplary extended release compositions in accordance with the present invention can provide a substantially level release of the clavulanate up to about 8 to 10 hours after exposure to an aqueous solution.

Pharmaceutical compositions according to embodiments of the invention provide important uses and advantages. One advantage of the present invention is the stability of the active ingredients in the composition. Control of water content is a major issue in the formulation and storage of clavulanate containing compositions because clavulanate is hygroscopic and is unstable or hydrolyzed in water. According to the invention, use of lyophilization to prepare a stabilized immediate release or extended release composition provides unexpectedly enhanced stability, particularly when the clavulanate is combined with excipients prior to lyophilization.

According to embodiments of the present invention a freeze dried composition of clavulanate can be used that includes: (1) forming a clavulanate composition by mixing clavulanate with at least one excipient; (2) freezing a quantity of the clavulanate composition₌, e.g., clavulanate, at 0° C. or below until converted into a frozen solid; and (3) dehydrating the clavulanate composition in an airtight container. The dehydrated (lyophilized) composition, including the drug, in powdered form can be mixed with other excipients before being compressed into tablets or prepared as sized beads.

The moisture content of the final dry formulation is low. The various embodiments set forth herein will have a final moisture content not exceeding about 10% (by weight), not exceeding about 5%, or not exceeding about 4%, or even lower. Dry formulations according to such embodiments of the invention are highly storage stable for extended periods, such as, for example, stable for about 30 days, about 60 days or about 90 days at conditions such as 25° C. and 60% relative humidity or 30° C. and 65% relative humidity. Upon dilution with the appropriate liquid, they are fully potent at substantially their stated initial dosage.

In some embodiments of the invention, the formulations are prepared by dry blending a polymer, for example a matrix such as Eudragit (anionic copolymers of methacrylic acid and ethyl acrylate), a binder/diluent such as Maltrin M50 and/or a disintegrating agent such as Pharmaburst, filler, clavulanate, and other excipients (see examples), followed by granulating the mixture using water until proper granulation is obtained. The granulation is done by methods known in the art. The wet granules are freeze dried in a freeze dryer, sifted and ground to appropriate size. Lubricating agents can be mixed with the dried granulation to obtain the final formulation. As clavulanate is hygroscopic and labile in water, it is necessary to minimize the time mixture remains wet, for example, the processing time from weighing and granulation to freeze drying can be about 1 hr.

The compositions of the invention can be administered orally in the form of tablets or capsules. The tablets can be prepared by techniques known in the art and contain a therapeutically useful amount of clavulanate and such excipients as necessary to form the tablet by such techniques. Placebo particles can also prepared without clavulanate but with same composition.

Pharmacokinetic Study

The bioavailability study for the formulations of the invention was measured by administering the immediate or extended formulation in a tablet form to healthy subjects and measuring the levels of clavulanate in the plasma at different time intervals over a period of twenty four hours. Plasma samples were assayed for clavulanate by BAS Analytics (West Lafayette, Ind.) using a validated high performance liquid chromatographic procedure similar to that described in the literature. See for example, Chu S-Y, et al., “Simultaneous determination of clarithromycin and 14(R)-hydroxyclarithromycin in plasma and urine using high performance liquid chromatography with electrochemical detection”, J. Chromatography, 571, pp 199-208 (1991).

### EXAMPLES

The following examples are for purpose of illustration only and are not intended to limit the scope of the appended claims.

### Example 1

### Preparation of Clavulanate Tablets

### Example 1A

### Preparation of Immediate Release Clavulanate Tablet Using Potassium Clavulanate Powder

Exemplary description of tablet preparation process: A wet granulation tablet formulation process has been discovered where water is included in a granulation step, followed by drying to obtain granules of low water content (<3%). The dried formulation is non-hygroscopic compared with prior art formulations, but maintains equivalent physical characteristics (for example, dissolution, disintegration, bioavailability and other physical properties) of the tablet prepared therefrom. The tablet preparation was carried out by granulating the clavulanate with water in the presence of binder/diluent. For the preparation of sample C, Maltrin M150 (130 g) was dissolved in purified water and potassium clavulanate (API; 59.5 g) was added. Prosolve SMCC-50 (490.5 g), Pharmaburst (130.0 g), L HPC LH-11 (120.0 g), Acdisol (20.0 g) and stearate acid (50 g) were weighed and mixed in a bag by shaking and rotating the bag. The mixture was transferred to the bowl of a Hobart mixer and the API/Maltrin M150 solution was added to the mixture with stirring for 10 minutes. After wet massing was completed, the contents of the bowl of the Hobart mixer were transferred into an extruder and extruded. The extrudate was placed into the spheronizer and the spheronized material was collected in a bag and lyophilized in a gortex-lyoguard tray. The dried material was screened and compressed into tablets or prepared into sized beads. Sample A and B were prepared in the same way as sample C.

### Example 1B

### Preparation of Immediate Release Clavulanate Tablet Using Clavitesse™

For the preparation of sample D, Clavitesse™ (API; 50.6 g), Prosolve SMCC 50 (213.4 g), Pharmaburst (100.0 g), Acdisol (8.0 g), Cabosil (8.0 g) and magnesium stearate (20.0 g) were weighed and lyophilized overnight in a gortex-lyoguard tray at 2-8° C. On the next day, the API, Prosolve SMCC 50, Pharmaburst and Acdisol were mixed in a bag, screened through # 40 mesh, unloaded into a V blender and mixed for 7 minutes. The mixture was screened again and mixed in the V blender for 4 min. The Cabosil and magnesium stearate were screened and mixed with the mixture containing API in the V blender for 4 min. The blend was lyophilized overnight in a gortex-lyoguard tray. The material was compressed into tablets and tablets were lyophilized in the gortex-lyoguard tray and packaged. Sample E was prepared in the same way as sample D.

### Example 1C

### Preparation of Extended Release Clavulanate Tablet Using Clavitesse™

For the preparation of sample F, suitable amounts of Clavitesse (API; 41.07 g), Methocell K100LV Prem CR (90.0 g), Isomalt (83.55 g), Avicel PH-112 (80.04 g), Cabosil (1.5 g), Talc (2.4 g) and magnesium stearate (1.5 g) were weighed and dried in Freeze dryer overnight with application in a gortex-lyoguard tray at 2-8° C. Each ingredient was screened and collected in a separate bag. API and Methocel K100LV Prem CR were loaded into a V blender, mixed, screened through a suitable sieve and mixing was continued. Avicel PH-112 and Isomalt were added to the mixture and mixed. The resulting mixture was screened and mixed again. Cabosil and Talc were mixed and added into the mixture and mixed. Magnesium stearate was mixed with the mixture in the V blender. The final blend was freeze dried overnight in a gortex-lyoguard tray and compressed into tablets or prepared into sized beads. Tablets were compressed at higher hardness for extended release coating. Tablets or beads were coated with delay release polymer, Eudragit.

### Example 1D

### Preparation of Extended Release Clavulanate Tablet Using Potassium Clavulanate Powder

For the preparation of extended release tablet using potassium clavulanate, Sample G, potassium clavulanate (API; 20.69 g) was screened through # 60 mesh and other excipients, Methocel K100LV Prem CR (90.02 g), Isomalt (83.56 g), Avicel PH-112 (100.41 g), Cabosil (1.52 g), Talc (2.4 g) and magnesium stearate (1.5 g), were screened through # 40 mesh. Each ingredient was collected in a separate bag. The API and Methocel K100LV Prem CR were loaded into a V blender and mixed for 5 minutes. The mixture was screened and mixed for 5 additional minutes. The Avicel PH-112 and Isomalt were added to the mixture and mixed in the V blender for 5 minutes. The resulting mixture was screened and mixed for 5 additional minutes. The Cabosil and Talc were mixed and loaded into the mixture and then the resulting mixture was mixed for 2 minutes. Finally, magnesium stearate was mixed with the mixture in the V blender for 3 minutes and the final blend was lyophilized overnight in the gortex-lyoguard tray and then compressed into tablets or prepared into sized beads. Tablets were compressed at higher hardness for extended release coating. Tablets or beads were coated with delay release polymer, Eudragit. Sample H and I were prepared in the same way with sample G.

### Example 2

### Assay of Clavulanate

The clavulanate content of the prepared pharmaceutical composition was measured by Waters HPLC (high performance liquid chromatography) system (column: μBondapack-NH₂ (10 μm) 300 mm×3.9 mm, Mobile phase: CH₃CN:pH 5.2 KH₂PO₄=65:35, Flow rate: 1.0 ml/min) using the following procedure: About 10 tablets were accurately weighed and grinded, 100 ml of water added and the mixture sonicated for 20 min. After dilution with water, a portion of solution was filtered and injected into HPLC. The major peak was identified by the retention time of the sample that corresponded to the chromatogram of the standard preparation by HPLC. The % clavulanate was calculated based on analyte response factor compared to the response factor of the reference standard.

Linearity of clavulanate standard curve was verified at 25, 50, 75, 100, 125, 150% of reference standard at nominal concentration of 0.01 mg/ml. R² was 0.9998. At nominal concentration of 0.01 mg/ml of clavulanate, precision was verified using six samples with percent of RSD 1.4. Accuracy was determined by preparing, in triplicate, and analyzing spiked placebo blends at 50%, 100%, and 150% of 0.01 mg/ml.

### Example 3

### Exemplary Formulation and Characteristics

The following experiments describe tablet formulation designed as immediate release (IR) tablet and extended release (ER) tablet with different doses. The following table also represents the physical properties of tablets according to the present formulation.

### Example 3A

### Immediate Release Composition Using Potassium Clavulanate

Immediate release compositions were prepared from potassium clavulanate powder and excipients as shown in Table 1 using the method described above.

TABLE 1

| | | Sample A, | Sample B, | Sample C, |
|---|---|---|---|---|
| Ingredient (mg) | Function | 0.1 mg/tablet | 0.3 mg/tablet | 5 mg/tablet |
| Potassium Clavulanate | API* | 0.1 | 0.357 | 5.95 |
| Maltrin M150 | Binder/diluent | 15 | 15 | 13 |
| Prosolve SMCC 50 | Filler | 50 | 50 | 49.05 |
| Pharmaburst | Disintegrating agent | 15 | 15 | 13 |
| L HPC LH-11 | Disintegrating agent | 15 | 15 | 12 |
| Acdisol | Disintegrating agent | 0.1 | 0.1 | 2 |
| Stearic acid | Lubricant | 4.8 | 4.543 | 5 |

API*: Active pharmaceutical ingredient.

Table 2 summarizes the characteristics of immediate release tablet using potassium clavulanate powder. Sample C tablet showed excellent stability, containing 94.4% of potassium clavulanate after 1 week at 2-8° C.

TABLE 2

| | | Sample A, | Sample B, | Sample C, |
|---|---|---|---|---|
| Parameter | Unit | 0.1 mg/tablet | 0.3 mg/tablet | 5 mg/tablet |
| Weight | mg | 106 | 106 | 101 |
| Hardness | KP | 5 | 5 | 3-5 |
| Thickness | mm | 0.155 | 0.155 | 3.6-3.8 |
| Disintegration Time | sec | 15 | 15 | 20 |
| Assay | % | 95.3 | 95.3 | 89.4-92.9% |
| 1 Week Assay 2-8° C. | % | — | — | 94.4 |
| Content Uniformity | RSD | 2.5 | 2.6 | 1 |
| Dissolution | % dissolved | — | 98% in 5 min | 89% in 5 min |
| Moisture Content-Final | % | — | 0.91 | 3.14 |

### Example 3B

### Immediate Release Composition Using Clavitesse™

Immediate release compositions comprising 5 mg of clavulanate were prepared using Clavitesse™ as shown in Table 3.

TABLE 3

| | | Sample D, | Sample E, |
|---|---|---|---|
| Ingredient (mg) | Function | 5 mg/tablet | 5 mg/tablet |
| 1:1 mixture of potassium | API* | 12.65 | — |
| clavulanate and | | | |
| microcrystalline cellulose | | | |
| 1:1 mixture of potassium | API* | — | 12.62 |
| clavulanate and silicon | | | |
| dioxide | | | |
| Prosolve SMCC 50 | Filler | 53.35 | 53.38 |
| Pharmaburst | Disintegrating | 25 | 25 |
| | agent | | |
| Acdisol | Disintegrating | 2 | 2 |
| | agent | | |
| Cabosil | Flow enhancer/ | 2 | 2 |
| | moisture | | |
| | protectant | | |
| Magnesium stearate | Lubricant | 5 | 5 |

API*: Active pharmaceutical ingredient.

Table 4 summarizes the characteristics of immediate release tablet using Clavitesse™.

TABLE 4

| | | Sample D, | Sample E, |
|---|---|---|---|
| Parameter | Unit | 5 mg/tablet | 5 mg/tablet |
| Weight | mg | 103-104 | 108 |
| Hardness | KP | 5-7 | 5-7 |
| Disintegration Time | min | <1 min | <2 min |
| Moisture content | % | 3.24 | 3.40 |

### Example 3C

### Extended Release Composition Using Clavitesse™ and Potassium Clavulanate Powder

Extended release compositions were prepared using Clavitesse™ or potassium clavulanate powder as shown in Table 5.

TABLE 5

| | | Sample F, | Sample G, | Sample H, | Sample I, |
|---|---|---|---|---|---|
| Ingredient (mg) | Function | 5 mg/tablet | 5 mg/tablet | 0.3 mg/tablet | 1.0 mg/tablet |
| 1:1 Mixture of potassium | API* | 13.69 | — | | |
| clavulanate and | | | | | |
| microcrystalline cellulose | | | | | |
| Potassium clavulanate | API* | — | 6.894 | 0.357 | 1.19 |
| Klucel LF | Matrix | | | 6 | — |
| (Hydroxypropylcellulose) | | | | | |
| Methocel K100 Prem-M | Matrix | | | — | 37 |
| Eudragit RS PO powder | Matrix | | | 20 | — |
| Methocel K100LV Prem | Matrix | 30.0 | 30.0 | | |
| CR | | | | | |
| Anhydrous lactose | Filler | | | 30 | — |
| Avicel PH-112 | Filler | 26.67 | 27.85 | 41.24 | — |
| Avicel PH-113 | Filler | | | — | 20 |
| Isomalt | Filler | 27.85 | 33.47 | — | 40 |
| Cabosil | Glidant | 0.5 | 0.5 | 0.8 | 0.5 |
| Magnesium stearate | Lubricant | 0.5 | 0.5 | 1.6 | 0.5 |
| Talc | Lubricant | 0.8 | 0.8 | — | 0.8 |
| Total | | 100 mg | 100 mg | 100 mg | 100 mg |

API*: Active pharmaceutical ingredient.

Table 6 summarizes the characteristics of extended release tablet using Clavitesse™ and potassium clavulanate powder

TABLE 6

| | | | | | Sample I, |
|---|---|---|---|---|---|
| | | Sample F, | Sample G, | Sample H, | 1.0 |
| Parameter | Unit | 5 mg/tablet | 5 mg/tablet | 0.3 mg/tablet | mg/tablet |
| Weight | mg | 99.9-102.4 | 92.0-108.3 | 104-105 | 108 |
| Hardness | KP | 9.9-14.0 | — | 7-9 | 10 |
| Assay | % | 105.9 | 96.2 | 0.756 | 3.44 |

### Example 4

### In Vitro Dissolution Studies

Tablets were placed in the 500 ml of solvent (deionized water for immediate release tablets; pH 1.2 solution for first 2 hrs and then pH 7.0 of citrate buffer for the next 8 hrs for extended release tablets). The mixture was swirled at 100 rpm and at 37° C. and a sample periodically collected and tested for the amount of dissolved clavulanate by HPLC.

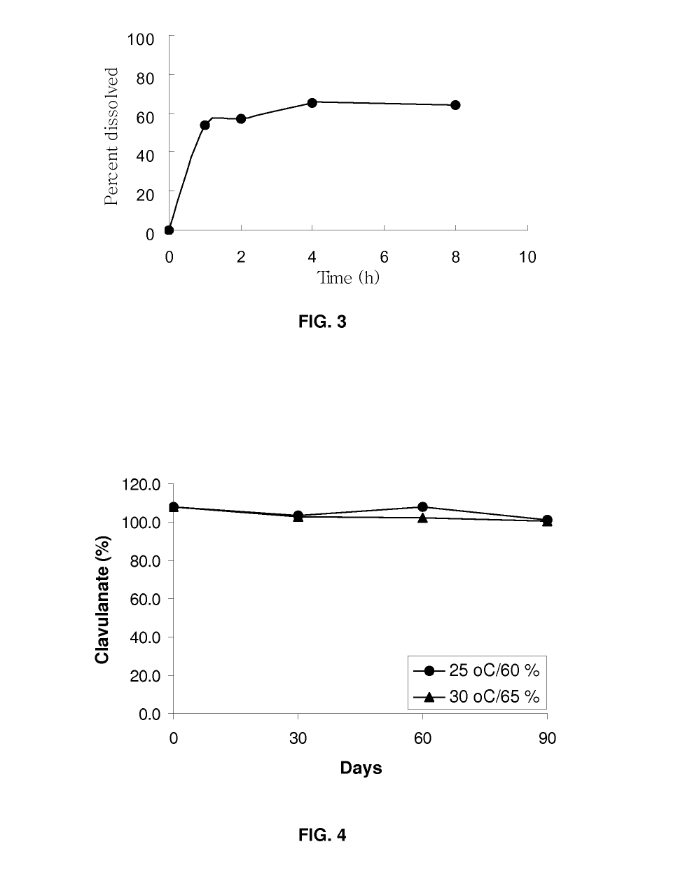

The results are shown in FIGS. 1-3. FIG. 1 is a graph showing the in vitro dissolution profiles of clavulanate immediate-release formulations of Sample B and Sample C. As shown in FIG. 1, 90% or more of clavulanate in the immediate release tablet was dissolved within 15 min after exposure to the aqueous solution. FIG. 2 is a graph showing the in vitro dissolution profile of the clavulanate extended-release formulation of Sample F. FIG. 3 is a graph showing the in vitro dissolution profile of the clavulanate extended-release formulation of Sample I. As shown in FIGS. 2 and 3, the total dose of clavulanate in the extended release tablet was slowly released via erosion and dissolution mechanisms over a period of at least about 8 to 10 hours. Release of clavulanate in the extended release form was not detected in pH 1.2 solution.

### Example 5

### Stability Test

Potassium clavulanate in its solid form is both hygroscopic and unstable in the presence of water vapor. A stability study of clavulanate was conducted with monitoring by chromatographic methods. The static or equilibrium approach was approached by storing samples in chambers at different relative humidity in an attempt to generate a sorption isotherm. The sorption isotherm represents the quantitative relationship between the equilibrium moisture content and relative humidity (RH) in the atmosphere. Table 7 shows the change of the water content in potassium clavulanate powder after exposed to the different humidity conditions.

TABLE 7

| | | Moisture Content (%) | Moisture Content (%) |
|---|---|---|---|
| Time | % RH | (g H₂O/g wet solid) | (g H₂O/g dry solid) |
| 96 hr | 33 | 0.708 | 0.713 |
| | 35 | 0.733 | 0.737 |
| | 37 | 0.842 | 0.848 |
| | 39 | 1.264 | 1.280 |
| | 41 | 1.542 | 1.566 |
| | 43 | 3.976 | 4.140 |
| | 45 | 4.778 | 5.018 |
| | 47 | 12.823 | 14.708 |

As shown in Table 7, potassium clavulanate has relatively low moisture content (<1% on a dry weight basis) when exposed to about 35% or less of relative humidity for 96 hr. However, it appears that deliquescence would eventually occur at any humidity above about 40% relative humidity. Moisture absorption by dry potassium clavulanate exposed to about 50% relative humidity occurs at a rate of approximately 1.44% per hour.

Potassium clavulanate is an exceptionally difficult material to formulate, being extremely moisture and heat sensitive. Degradation readily occurs in the presence of water and aqueous media. Several methods were tested to find a suitable condition for removing moisture after wet granulation that keeps the active ingredient clavulanate intact. The material in sample C was prepared by wet granulation and spheronized. The moisture containing spheronized formulation was transferred to trays and subjected to different storage conditions for the removal of moisture.

As summarized in Table 8, storage at 30° C. for 69 hr (storage 1), or storage at 45° C. for 75 hr (storage 2), resulted in the degradation of potassium clavulanate up to 45% and 60% respectively. Drying in a fluid bed system resulted in degradation of the clavulanate by 13% in only 1.5 hr. These data suggest that potassium clavulanate is also temperature sensitive. Lyophilization retained 97% of the active ingredient after 21 hrs of the freeze drying process. The results in Table 8 show that lyophilization of clavulanate can be used to reduce the content of moisture in a clavulanate formulation and increase the stability of the formulation.

TABLE 8

| Method | Temp (° C.) | Time (hr) | Clavulanate (%) |
|---|---|---|---|
| Storage 1 | 30 | 69 | 55 |
| Storage 2 | 45 | 75 | 40 |
| Fluid bed | 40 | 1.5 | 87 |
| Freeze dry | Sub-zero | 21 | 97 |

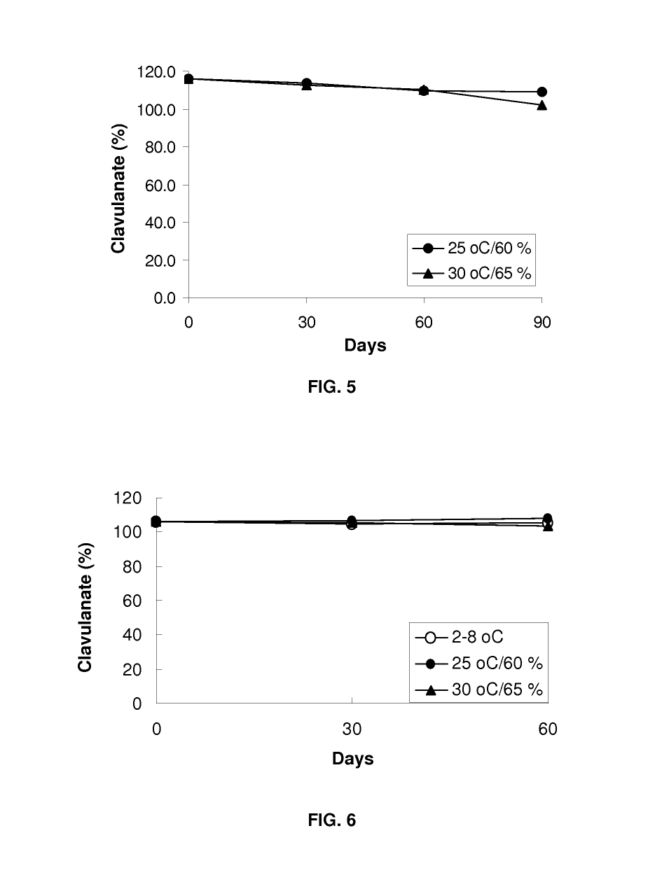

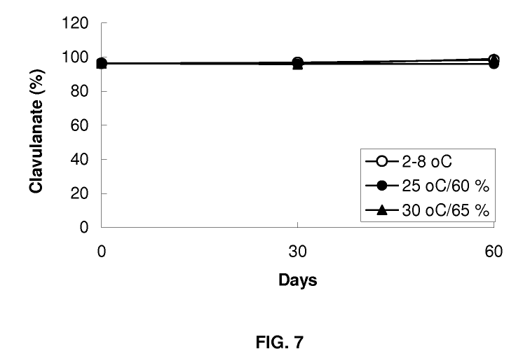

Stability of immediate release tablets prepared from Clavitesse™, Sample D and Sample E, was evaluated for up to 3 months. FIG. 4 is a graph showing the stability of Sample D (5 mg/tablet of 1:1 mixture of potassium clavulanate and microcrystalline cellulose) at 25° C./60% humidity and 30° C./65% humidity. FIG. 5 is a graph showing the stability of Sample E (5 mg/tablet of 1:1 mixture of potassium clavulanate and silicon dioxide) at 25° C./60% humidity and 30° C./65% humidity. As shown in Table 4 and in FIGS. 4 and 5, both tablets prepared according to Samples D and Sample E initially contained less than 4%-moisture and were degraded less than 7% at 25° C./60% humidity, a relative high humidity condition for clavulanate. Stability of extended release tablets prepared from Clavitesse™, Samples F and G were evaluated for up to 2 months. FIG. 6 is a graph showing the stability of Sample F (5 mg/tablet of 1:1 mixture of potassium clavulanate and microcrystalline cellulose) at 2-8° C., 25° C./60% humidity and 30° C./65% humidity. FIG. 7 is a graph of the stability of Sample G (5 mg/tablet) at 2-8° C., 25° C./60% humidity and 30° C./65% humidity. As shown in Table 5 and in FIGS. 6 and 7, the tablets prepared according to Samples F and G initially contained less than 4%-moisture and were degraded less than 1.6% at 30° C./65% humidity, a relative high humidity condition for clavulanate. Therefore it appears that microcrystalline cellulose or silicon dioxide in Clavitesse™ may further contribute the increase of stability of potassium clavulanate by capturing the moisture in a tablet.

### Example 6

### Pharmacokinetic Study

The amount of clavulanate in the plasma of beagle dogs was measured by LC/MS/MS method. The chromatographic separation of the analytes was performed on a reverse-phase PLRP-S polymeric column. The retention time of potassium clavulanate and tazobactam (reference compound) were 8.51 and 8.54 min, respectively. The overall chromatographic run time was 25 min. The M/S analysis was performed on an Applied Biosystems' API 2000 triple-quardrupole mass spectrometer by multiple reaction monitoring in negative electrospray ionization mode. The mass spectral data were analyzed by Analyst 1.4.1 (Applied Biosystems). The pharmacokinetic analysis was conducted by using PK Solutions 2.0 (Summit Research Services).

### Example 6A

Oral Administration of Immediate Release (IR) Tablet in Male Beagle Dogs

Three male Beagle dogs were used throughout the study in a cross-over design with washout period between treatments. The dogs were given the test substances as IR tablet of Example 3A via oral routes with no shorter than 24 hr washout period between dosing. The animals were fasted overnight before the administration of the test substance and fed 4 hr post-dosing. During all the treatments, blood samples (1.5 ml) were withdrawn from the cephalic vein by venipuncture into heparinized tubes at 0, 5, 15, 30 min, 1, 1.5, 2, 2.5, 3, 4, 6, 9 and 12 hr after dosing. Plasma was obtained via centrifugation at 3,000 rpm for 10 min and analyzed by an LC-MS/MS system. The associated mean pharmacokinetic parameters are provided in Table 9.

### Example 6B

### IV Administration of Potassium Clavulanate Solution in Male Beagle Dogs

Three male beagle dogs were used throughout the study in a cross-over design with washout period between treatments. The dogs were given the test substances as aqueous solution via intravenous routes with no shorter than 24 hr washout period between dosing. The animals were fasted overnight before the administration of the test substance and fed 4 hr post-dosing. During all the treatments, blood samples (1.5 ml) were withdrawn from the cephalic vein by venipuncture into heparinized tubes at 0, 5, 15, 30 min, 1, 1.5, 2, 2.5, 3, 4, 6, 9 and 12 hr after dosing. Plasma was obtained via centrifugation at 3,000 rpm for 10 min and analyzed by an LC-MS/MS system. The associated mean pharmacokinetic parameters are provided in Table 9.

### Example 6C

### Oral administration of Extended Release (ER) Tablet in Male Beagle Dogs

Four male beagle dogs were used throughout the study in a cross-over design with washout period between treatments. The dogs were given the test substances as ER tablet of Example 3C via oral routes with no shorter than 24 hr washout period between dosing. The animals were fasted overnight before the administration of the test substance and fed 4 hr post-dosing. During all the treatments, blood samples (1.5 ml) were withdrawn from the cephalic vein by venipuncture into heparinized tubes at 1, 2, 3, 4, 5, 6, 7, 8, 9, 10, 11 and 12 hr after dosing. Plasma was obtained via centrifugation at 3,000 rpm for 10 min and analyzed by an LC-MS/MS system. The associated mean pharmacokinetic parameters are provided in Table 9.

TABLE 9

| | | | | | | | | Oral | |
|---|---|---|---|---|---|---|---|---|---|
| | | IV | | Oral (IR tablet) | | | | (ER tablet) | |
| PK Parameter* | | mean | SD | mean | SD | mean | SD | mean | SD |
| Dose | (mg) | 4.2 | — | 3.5 | — | 7.4 | — | 21.6 | — |
| Tₘₐₓ | (hr) | — | — | 1.2 | 0.3 | 1.2 | 0.3 | 2.8 | 1.0 |
| Cₘₐₓ | (ng/ml) | — | — | 125.8 | 80.0 | 413.7 | 127.9 | 821.3 | 492.7 |
| AUC0-t | (hr · ng/ml) | 684.4 | 74.6 | 175.6 | 101.8 | 498.4 | 70.8 | 1702.4 | 580.6 |
| CL | (l/hr) | 5.8 | 0.7 | | | | | | |
| Vd | (l) | 4.4 | 0.5 | — | — | — | — | — | — |
| Vss | (l) | 3.8 | 0.4 | — | — | — | — | — | — |
| t_{1/2} | (hr) | 0.52 | 0.02 | 0.49 | 0.09 | 0.46 | 0.02 | 1.9 | 1.5 |
| MRT_{inf} | (hr) | 0.65 | 0.01 | 1.6 | 0.1 | 1.7 | 0.3 | 3.4 | 1.5 |
| F | (%) | 100 | — | 29.9 | 14.7 | 41.4 | 4.7 | 45.4 | 15.5 |

*PK parameters: Tₘₐₓ: time to maximum concentration, Cₘₐₓ: maximal concentration, AUC: area under the curve, CL: clearance, Vd: volume of distribution, Vss: volume of distribution at steady state, t_{1/2}: half-life, MRT_{inf}: mean residence time, F: bioavailability

Potassium clavulanate was shown to be well absorbed in fasted animals, with an average bioavailability of 30˜41%, when given orally. The apparent terminal half-life was 0.5 hr.

The embodiments illustrated and discussed in this specification are intended only to teach those skilled in the art the best way known to the inventors to make and use the invention. Nothing in this specification should be considered as limiting the scope of the present invention. All examples presented are representative and non-limiting. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described.

What is claimed is:

1. A solid pharmaceutical composition comprising between about 10 μg and about 10 mg of a clavulanate in the presence of one or more pharmaceutically acceptable excipients;
wherein the clavulanate is selected from the group consisting of clavulanic acid, clavulanic acid derivatives or a pharmaceutically acceptable salt of clavulanic acid.

2. The pharmaceutical composition of claim 1, comprising or between about 0.01% and about 10% by weight of the clavulanate.

3. The pharmaceutical composition of claim 1, having a moisture content of less than about 4% of total weight of the pharmaceutical composition.

4. The pharmaceutical composition of claim 1, wherein the clavulanate is potassium clavulanate.

5. The pharmaceutical composition of claim 4, wherein the potassium clavulanate is provided as a powder or as a 1:1 mixture with silicon dioxide or microcrystalline cellulose.

6. The pharmaceutical composition of claim 1, wherein the formulation is an immediate-release composition which releases more than 80% of clavulanate from the tablet within approximately 30 minutes after administration.

7. The pharmaceutical composition of claim 6, further comprising from about 10% to about 20% by weight of a binder or diluent, about 45% to about 55% by weight of a filler, about 20% to about 40% by weight of a disintegrant and about 3% to about 6% by weight of a lubricant; wherein the potassium clavulanate is provided as a powder.

8. The pharmaceutical composition of claim 6, further comprising about 50% to about 60% of a filler, about 20% to about 30% of a disintegrant, about 0.5% to about 5% of a flow enhancer/moisture protectant and/or about 3% to about 6% of a lubricant;
wherein the potassium clavulanate is provided as a 1:1 mixture with silicon dioxide or microcrystalline cellulose.

9. The pharmaceutical composition of claim 1, wherein the formulation is an extended-release composition which releases the clavulanate for at least about 4 hours.

10. The pharmaceutical composition of claim 9, wherein the clavulanate is provided as potassium clavulanate powder or potassium clavulanate in a 1:1 mixture with microcrystalline cellulose.

11. The pharmaceutical composition of claim 10, further comprising about 20% to about 40% by weight of a matrix, about 50% to about 75% by weight of a filler, about 0.1% to about 1% by weight of a glidant and about 1% to about 2% by weight of a lubricant.

12. The pharmaceutical composition of claim 1, wherein the formulation is the form of a tablet, capsule, pill, troche or powder.

13. A method of making a solid pharmaceutical dosage form comprising the steps of:
providing a clavulanate selected from the group consisting of clavulanic acid, clavulanic acid derivatives or a pharmaceutically acceptable salt of clavulanic acid;
mixing the clavulanate with at least one excipient;
granulating the mixture of clavulanate and the at least one excipient; and
lyophilizing the granulated mixture of clavulanate and the at least one excipient.

14. The method of claim 13, wherein the granulating step comprises wet granulation.

15. The method of claim 13, wherein the clavulanate is potassium clavulanate.

16. The method of claim 15, wherein the potassium clavulanate is potassium clavulanate powder or potassium clavulanate as a 1:1 mixture with silicon dioxide or microcrystalline cellulose.

17. The method of claim 13, wherein the excipient is selected from the group consisting of a binder, a diluent, a filler, a disintegrant, a matrix, a filler, a glidant, a flow enhancer, a moisture protectant, and a lubricant.

18. The method of claim 13, further comprising forming the dosage form into a tablet or bead.

19. The method of claim 18, further comprising coating the tablet or beads with a delay-release polymer.

20. A solid pharmaceutical composition prepared by the process of
providing a clavulanate selected from the group consisting of clavulanic acid, clavulanic acid derivatives or a pharmaceutically acceptable salt of clavulanic acid;
mixing the clavulanate with at least one excipient;
granulating the mixture of clavulanate and the at least one excipient; and
lyophilizing the granulated mixture of clavulanate and the at least one excipient.

21. The pharmaceutical composition of claim 1, having a moisture content of less than 10% after storage at 25° C. and 60% relative humidity or 30° C. at 65% relative humidity.

22. A method of treatment comprising administering a pharmaceutical composition according to claim 1 that comprises an amount of clavulanate effective for the treatment of a disorder selected from sexual dysfunction and a neurological disorder.

23. The method of claim 22, wherein the composition is an extended release composition and the disorder is a neurological disorder selected from anxiety and depression.

24. The method of claim 23, wherein the composition is an immediate release composition and the disorder is sexual dysfunction.
